(19) United States
(12) Patent Application Publication
(10) Pub. No.: US 2004/0234961 A1
(43) Pub. Date: Nov. 25, 2004

(54) Telomere length determination and applications

(76) Inventors: Colleen A. Fordyce, San Francisco, CA (US); Jeffrey K. Griffith, Cedar Crest, NM (US)

Correspondence Address: MUETING, RAASCH & GEBHARDT, P.A., P.O. BOX 581415, MINNEAPOLIS, MN, 55458, US

(21) Appl. No.: 10/421,166

(22) Filed: Apr. 22, 2003

Related U.S. Application Data
(60) Provisional application No. 60/374,937, filed on Apr. 22, 2002.

(51) Int. Cl. C12Q 1/68

(52) U.S. Cl. 435/6

(57) ABSTRACT

Methods for determining the telomere content in a sample, based on a ratio of telomeric DNA to total DNA or a subset of total DNA. Methods for determining the prognosis of a patient with cancer, including breast or prostate cancer, by determining the telomere content in a sample, the sample obtained from either tumor tissue or coexisting histologically normal tissue.

### CROSS-REFERENCE TO RELATED APPLICATION

This application claims the benefit of the filing of U.S. Provisional Patent Application Serial No. 60/374,937, entitled Telomere Length Determination and Applications, filed on Apr. 22, 2002, and the specification thereof is incorporated herein by reference.

### BACKGROUND OF THE INVENTION

1. Field of the Invention (Technical Field)

The invention relates to methods to determine telomere content, and more specifically methods to determine the total quantity of telomeric sequences to total DNA, or to a subset of total DNA. The invention further relates to methods of determining cancerous and pre-cancerous tissues, such as for surgical procedures, methods of staging cancers and other diseases, and methods of selecting, monitoring, evaluating and determining the effectiveness of different methods of treating disease, including cancer and viral diseases.

2. Description of Related Art

Note that the following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

Telomeres are nucleoprotein complexes that protect the ends of chromosomes from fusion and degradation. (Allsopp, R. C., E. Chang, M. Kashefiaazam, E. I. Rogaev, M. A. Piatyszek, J. W. Shay and C. B. Harley. Telomere shortening is associated with cell division in vitro and in vivo. Exp. Cell Res. 220:194-200 (1995); Bechter, O. E., W. Eisterer, G. Pall, W. Hilbe, T. Kuhr and J. Thaler. Telomere length and telomerase activity predict survival in patients with B cell chronic lymphocytic leukemia. Cancer Res. 58(21):4918-4922 (1998); Moyzis, R. K., J. M. Buckingham, L. S. Cram, M. Dani, L. L., Deaven, M. D. Jones, J. Meyne, R. L. Ratliff and J. R. Wu. A highly conserved repetitive DNA sequence, (TTAGGG)ₙ, present at the telomeres of human chromosomes. Proc. Natl. Acad. Sci. USA 85:6622-6626 (1988).) Since telomeres are shortened each time a cell divides, tumor cells typically have shorter telomeres than paired normal cells. Normal somatic cells have telomeric nucleoprotein complexes some 10-12 Kbp in length in somatic cells, but as small as 1-2 Kbp in rapidly growing cancer cells.

Reduced telomere DNA length has been associated with aneuploidy, metastasis, grade, disease recurrence and survival. To validate the potential prognostic significance of telomere DNA content, as well as use this marker system in patient diagnosis, it is necessary to measure telomere DNA content in relevant clinical specimens, particularly in biopsies.

In most cancers, currently available prognostic markers fail to differentiate between aggressive tumors and comparatively indolent or non-aggressive tumors. This problem is particularly acute with cancers such as breast and prostate cancers. For example, prognostic markers of breast cancer, including nodal status and tumor size, generally do not differentiate between aggressive tumors that have metastasized beyond the breast to the axial lymph nodes at the time of diagnosis and indolent tumors that have not metastasized. Accordingly, many women with breast cancer receive adjuvant chemotherapies and hormonal therapies that are, in many instances, unnecessary. Although adjuvant therapies improve overall survival, particularly of high-risk patients, the consequences and complications of these therapies, which include fatigue, nausea, vomiting, alopecia, myelosuppression, cardiotoxicity and the development of secondary malignancies, including leukemia, are severe and markedly reduce the patients' quality of life. Therefore, it is necessary to identify prognostic markers that reliably predict the likelihood of disease recurrence in women with breast cancer so as to differentiate between the subsets of women that will benefit from adjuvant therapy from those who can be spared unnecessary side effects. The same prognostic and therapeutic challenges are present with prostate cancer, and to an extent, with virtually all cancers.

Progression of many cancers is believed to be a multi-step, evolutionary process. The acquisition of an initial mutation confers a proliferative advantage to preneoplastic cells, ultimately allowing them to accumulate genetic alterations that affect critical phenotypes, including changes in cell-cell adhesion, angiogenic and invasive potential, and hormone independence. These variant cells can be retained in the population and expanded by inhibition of apoptosis and the reactivation of telomerase. In this model, successive rounds of mutation, Darwinian selection and clonal expansion culminate in the emergence of tumors with genetic programs necessary for invasion, extravasation and metastasis. The amplification or loss of a critical genes or genes is one mechanism by which the genetic programs of cells are altered during cancer progression. For example, loss of heterozygosity is common in cancer, including ductal carcinoma in situ, an immature form of breast cancer. Loss of heterozygosity also occurs frequently in histologically normal tissue coexisting with breast tumors and exists, in some instances, in breast tissue from women with benign breast disease. These observations are consistent with the hypothesis that genetic instability arises while breast tissues are phenotypically normal and seemingly disease free and may be an early event in breast cancer tumorigenesis.

One cause of genomic instability is dysfunctional telomeres. Extensive loss of telomeric DNA results in complex types of genomic abnormalities, including loss of heterozygosity, gene truncation and amplification, and aneupliody. During DNA replication, binding of the DNA polymerase complex to the terminal nucleotides of the leading DNA strand sterically inhibits their replication. Thus, telomeres are shortened each time the cell divides. Telomere dysfunction may also be a consequence of double-strand DNA breaks or mutations affecting the functions of any of the several proteins required for telomere maintenance. Since telomerase, the reverse transcriptase that elongates telomeres, is not expressed in most somatic cells, telomere loss is believed to be cumulative, irreversible and a reflection of a cell's proliferative history. Cancer cells, with extensive proliferative histories, typically have shorter telomeres and are more likely to be genetically unstable.

A number of methods have been utilized to determine telomere length or content. Based on the repeat sequence TTAGGG, a number of probe methods have been developed, such as utilizing a Southern blot or other methodologies incorporating electrophoresis, as described in part in U.S. Pat. No. 5,489,508. Other techniques for measuring telomere length have been developed, such as the primer-based methods, optionally utilizing polymerase chain reactions (PCR) to obtain sufficient sample, as disclosed in U.S. Pat. No. 5,834,193. U.S. Pat. No. 6,235,468 similarly discloses a primer-based method using PCR, wherein the invention resides in specific primers. Other primer-based methods include those disclosed in U.S. Pat. No. 5,695,932. The use of “telomeric repeat factors”, including nucleic acid sequences which encode telomeric repeat factors, is also disclosed and utilized in various assay systems, as disclosed in U.S. Pat. No. 6,297,356. U.S. Pat. No. 6,297,356.

An alternative method of telomere detection is disclosed in U.S. Pat. No. 5,871,926, involving the use of measurement of telomeric content, as opposed to length, based on the ratio of telomeric to centromeric DNA present in the tissue. In one embodiment, a “slot-blot” assay is employed with slot-blotted DNA hybridized with telomere- and centromere-specific oligonucleotide probes. In contrast to the commonly used Southern blotting method of measuring terminal telomere restriction fragments, this slot blot assay requires less than 0.1 μg of genomic DNA and is insensitive to DNA breakage. However, while this method represents an improvement over other prior art methods, the limit of sensitivity with this method, about 30 ng of total DNA, is insufficient for use with biopsy and other limited or scant tissue samples. In addition to lacking the sensitivity required for use with very small DNA samples, this method takes 5-8 days to complete, and relies upon hybridization of a second, centromere-specific, oligonucleotide to normalize for DNA quantity/load and reactivity. U.S. Pat. No. 5,871,926 also further discloses that the ratio of telomere to centromere content is directly proportional to the telomere restriction fragment length determined by Southern blot analysis and is not affected by interstitial or subtelomeric DNA sequences, making the ratio of telomere content to centromere content a surrogate for telomere length.

There thus exists in the art a need for a method to determine telomere content in small samples, including samples containing less than about 30 ng of DNA. Similarly, there exists a need for an assay system that is simple, relatively quick, and highly reliable, in order to permit routine determination of telomere content. There also exists in the art a need for methods that provide relevant clinical information, such as determination of prognostic information with relevance to clinical outcomes and desired clinical treatment.

### BRIEF SUMMARY OF THE INVENTION

In one embodiment, the invention provides an assay for measuring telomere content as a function of total DNA in a sample of DNA, which assay includes the steps of quantitating telomere content and total DNA content of the sample, and calculating the amount of telomere present relative to total DNA. The step of quantitating the total DNA content of the sample can further include adding a fluorescent dye capable of forming a fluorescent dye-DNA complex to the sample, measuring the amount of bound fluorescent dye, and correlating the amount of measured bound fluorescent dye relative to a control of known total DNA to determine total DNA. The step of quantitating telomere content can include adding a labeled probe having a sequence complementary to a telomere repeat sequence to the sample, and measuring the amount of labeled probe. The assay can include a slot-blot assay step, such as a step wherein slot-blotted DNA is hybridized with a telomere-specific oligonucleotide probe. The DNA can be obtained from paraffin-fixed tissue or formalin-fixed tissue, as well as frozen, fresh or dried tissue. The assay can employ a sample of DNA which contains less than about 50 ng of DNA, optionally less than 10 ng of DNA, and further optionally less than 5 ng of DNA.

The invention further includes a method for measuring telomere content as a function of total DNA in a sample including DNA, the method including the steps of:

adding a fluorescent dye capable of forming a fluorescent dye-DNA complex to the sample;

measuring the amount of bound fluorescent dye;

correlating the amount of measured bound fluorescent dye relative to a control of known total DNA to determine total DNA in the sample;

adding a labeled probe having a sequence complementary to a telomere repeat sequence to the sample;

measuring the amount of labeled probe; and

correlating the amount of bound probe measured relative to total DNA.

In this method, the fluorescent dye can be added to a first quantity of the sample and the labeled probe added to a second quantity of the sample.

The invention further includes a method for measuring telomere content as a function of total DNA in a sample including DNA, the method including the steps of:

adding a fluorescent dye forming a fluorescent dye-DNA complex to two or more diluted quantities of the sample and separately to at least one first quantity of a standard including a known amount of DNA;

measuring the amount of bound fluorescent dye in the two or more diluted quantities of the sample and the at least one first quantity of standard;

determining the total DNA in the sample by comparing the fluorescent intensity of the diluted quantities of the sample to the at least one quantity of the standard including a known amount of DNA;

adding a labeled probe having a sequence complementary to a telomere repeat sequence to two or more quantities of the sample and separately to at least one second quantity of a standard including a known telomere content as a function of total DNA;

measuring the amount of labeled probe in the two or more diluted quantities of the sample and the at least one second quantity of standard; and

determining telomere content in the sample as a function of total DNA by comparing the fluorescent intensity of two or more quantities of the sample as a function of total DNA to the fluorescent intensity of the at least one second quantity of standard.

In yet another embodiment, the invention provides a method for measuring telomere content of tumor cells in a first sample including DNA from a mixed population of cells including tumor cells and histologically normal cells, including the steps of:

determining the telomere content of the first sample as a function of total DNA;

obtaining a second sample of histologically normal cells and determining the total telomere content of the second sample as a function of total DNA;

determining the percentage of the total cells of the first sample that are histologically normal;

calculating the telomere content of tumor cells in the first sample by means of the formula:

(TC_{T})=[TC_{obs}−(TC_{N})(% N)]/(% T)

wherein TC_{T} is the telomere content of the tumor cells of the first sample as a function of total DNA, TC_{obs} is the determined total telomere content of the first sample as a function of total DNA, TC_{N} is the determined telomere content in the second sample of histologically normal cells, % N is the percentage of histologically normal cells in the first sample and % T is the percentage tumor cells in the first sample. In this method, the total telomere content of the first sample as a function of total DNA can be determined by quantitating telomere content and total DNA content of the first sample, and calculating the amount of telomere present relative to total DNA. In one embodiment, the first sample and the second sample are obtained from the same patient.

In yet another embodiment, the invention provides a method for determining the prognosis of a patient with cancer, the method including the steps of obtaining a tumor specimen from the patient, determining the telomere content of the tumor specimen as a function of total DNA, and categorizing the determined telomere content with respect to a mean telomeric content. In this method, determining the telomere content of the tumor specimen as a function of total DNA further optionally includes purifying the sample to isolate DNA, quantitating telomere content and total DNA content of the sample, and calculating the amount of telomere present relative to total DNA. Quantitating the total DNA content of the sample can include adding a fluorescent dye capable of forming a fluorescent dye-DNA complex to the isolated DNA, measuring the amount of bound fluorescent dye, and correlating the amount of measured bound fluorescent dye relative to a control of known total DNA to determine total DNA. Quantitating telomere content can include adding a labeled probe having a sequence complementary to a telomere repeat sequence to the isolated DNA, and measuring the amount of labeled probe. The method may be employed less than about 50 ng of DNA, optionally less than about 10 ng of DNA, and further optionally less than about 5 ng of DNA. This method may be employed with any cancer, including breast cancer and prostate cancer.

In a further embodiment, the invention provides a method for determining the prognosis of a patient with cancer, the method comprising obtaining a coexisting histologically normal tissue sample from the patient and determining the telomere content of the coexisting histologically normal sample as a function of total DNA. In this method, the determined telomere content of the coexisting histologically normal tissue can be categorized with respect to a mean telomeric content. Determining the telomere content of the coexisting histologically normal tissue sample as a function of total DNA can include purifying the sample to isolate DNA, quantitating telomere content and total DNA content of the sample, and calculating the amount of telomere present relative to total DNA, utilizing methods as set forth above. The coexisting histologically normal tissue sample can be obtained from a site at least about 1 cm distal from any histologically abnormal tissue. In another embodiment, the coexisting histologically normal tissue sample can be obtained from a site within about 5 cm of histologically abnormal tissue.

In a further embodiment, the invention provides a method of determining tissue boundaries in a surgical procedure to resect cancerous or pre-cancerous tissues in a patient, comprising determining the telomere content of tissue as a function of total DNA. In one aspect, determining the telomere content of tissue as a function of total DNA can include purifying the tissue to isolate DNA, quantitating telomere content and total DNA content of the tissue, and calculating the amount of telomere present relative to total DNA. Quantitating the total DNA content of the tissue can include adding a fluorescent dye capable of forming a fluorescent dye-DNA complex to the isolated DNA, measuring the amount of bound fluorescent dye, and correlating the amount of measured bound fluorescent dye relative to a control of known total DNA to determine total DNA. Quantitating telomere content can include adding a labeled probe having a sequence complementary to a telomere repeat sequence to the isolated DNA, and measuring the amount of labeled probe. However, other methods to determine telomere content, or optionally to directly measure telomere length or average telomere length, may also be employed. This method may be employed with any cancer, including breast cancer and prostate cancer.

A primary object of the present invention is to provide a method for determining telomere content, as a proxy for telomere length, as a function of total DNA in a sample.

Another object of the present invention is to provide a method that will reliable permit determination of telomere content as a function of total DNA, utilizing specimens with less than 100 ng of total DNA, preferably with less than about 50 ng of total DNA, more preferably with less than about 10 ng of total DNA, and most preferably with less than about 5 ng of total DNA.

Another object of the present invention is to provide a method for determining telomere content of mixed cell populations, where some cells are normal and other cells are not normal.

Another object of the present invention is to provide a method for determining telomere content that does not utilize a polymerase chain reaction.

Another object of the present invention is to provide a method for providing prognostic information with respect to carcinoma, particularly breast and prostate cancers, by determining the telomere content.

Another object of the present invention is to provide a method for providing prognostic information based on telomere content of histologically normal tissues proximal to histologically abnormal tissues.

A primary advantage of the present invention is that it provides a rapid, inexpensive and simple method of determining telomere content as a function of total DNA, in a format that is readily amenable to automated systems.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 depicts a representative slot blot utilized in the invention, showing placental, HeLa and five archival paraffin-embedded prostate tumor samples, with placenta and HeLa DNA concentrations ranging from 0-50 ng, with tumor DNA samples analyzed in triplicate at 40 ng.

FIG. 2 is a plot depicting the telomere contents in DNA admixtures, with HeLa and placenta DNAs mixed in 0%, 25%, 50%, 75% and 100% HeLa proportions, with data shown representing telomere DNA contents measured in 60 ng of total genomic DNA analyzed in triplicate, utilizing regression line and correlation coefficient analysis, with error bars representing standard deviations of the means.

FIG. 3 is a plot depicting the effect of formalin exposure on a telomere content assay, with human placenta placed in formalin for 0 to 8 hrs at room temperature, and telomere DNA contents thereafter measured; identical results were obtained from all time points, with data from 0 and 8 hrs shown.

FIG. 4 is a box plot graph showing tumors divided into two groups, disease free survival and breast cancer recurrence, as shown on the x-axis, and telomere content is shown on the y-axis.

FIG. 5 are box plot graphs for both tumor and coexisting histologically normal tissues in patients with breast cancer. For Kaplan-Meier graphs, tumor and coexisting histologically normal tissues were grouped according to telomere content of each group, greater than the median (high) or less than or equal to the median (low). P value was calculated using Log Rank analysis.

FIG. 6 are box plot graphs of telomere DNA content in a prostate cancer study, with panel A depicting prostate tumors divided into two groups, recurrent and disease-free (p=0.01); and panel B depicting prostate tumors divided into two high and low groups and analyzed as in FIG. 4 (p=0.04).

FIG. 7 is a plot of telomere DNA contents in somatic and tumor cells, with similar results obtained in three independent preparations of human DNA, including purified placenta DNA, HeLa DNA and human blood DNA obtained commercially, with in each instance a linear relationship between telomere DNA content and DNA input (R2=0.98-0.99), with the y-axis depicting relative units of telomere DNA content.

FIG. 8 is a plot of telomere DNA contents in sham needle core biopsies, demonstrating that telomere DNA contents were reliably measured in genomic DNA isolated from sham, needle core prostate biopsies with slopes, x-intercepts and correlation coefficients calculated by linear regression with the y-axis depicting relative units of telomere DNA content.

### DETAILED DESCRIPTION OF THE INVENTION

The invention described herein provides, in one embodiment, a method to measure telomere DNA context by means of a chemiluminescent assay, which assay can be employed with very low quantities of genomic DNA, on the order of 2 to 5 ng. In another embodiment, the invention provides a method for staging and determination of disease, the method including determination of the telomeric content of DNA. In yet another embodiment, the invention provides a measure for the efficacy of therapy by measurement of telomeric DNA.

The chemiluminescent slot blot assay for telomere DNA content has the sensitivity required for use with biopsy materials. Results obtained with DNA derived from human placental, HeLa, human peripheral blood lymphocytes, sham needle core prostate biopsies, archival prostatectomy tissues and archival breast cancer tissues demonstrate that telomere DNA content can be reliably and reproducibly measured in 5 ng, and sometimes in as little as 2 ng of genomic DNA. Sham needle core prostate biopsy and prostatectomy specimens processed in parallel produce comparable results. The contribution of truncated telomeres in admixtures containing as much as 75% normal placental DNA can be established. In addition, treatment of tissue with formalin prior to DNA purification does not decrease the efficacy of the assay.

The methods disclosed herein, including the chemiluminescent assay for telomere DNA content, is particularly well-suited for analysis of biopsy or microdissected specimens. The assay, including DNA purification and quantification, can be completed in two days and requires as little as 2-5 ng of genomic DNA, the equivalent of approximately 285-715 dipolid cells. This technique is at least ten times more sensitive and five times faster than existing methods. In addition, the assay is unaffected by standard formalin treatment and is informative for tumor tissues that contain up to 75% normal cells.

Telomere DNA contents determined by this method reflect the average telomere content in cells comprising the sample. The method does not provide information about the variability of telomere DNA content on chromosomes within or between individual cells. However, with the assay's improved sensitivity, telomere DNA content can be measured in microdissected or flow sorted tumor cell sub-populations.

Determination of Telomere Content. The methods disclosed herein include three discrete steps, which steps may be conducted in any feasible order, and may, as appropriate, be conducted simultaneously. First, the total DNA in the sample is determined. Second, the quantity of telomere DNA in the sample is detected. Third, the amount of telomere DNA relative to total DNA is determined. While the methods disclosed herein incorporate certain preferred embodiments relative to each step, it is to be emphasized that the methods disclosed herein are not dependent on any specific method for conducting each step. Thus, as will be apparent to one of skill in the art, methods different than those disclosed here may be employed for determining total DNA in a sample and for determining telomere DNA content in the sample. The description following is intended to be illustrative of methodologies that may be employed, and is not intended to provide an exhaustive description of methods, protocols, reagents or technologies that may be employed.

Quantitation of DNA Content. The sample or samples on which the assays are performed may be obtained by any means. Examples include needle-core biopsies, surgical biopsies, excision of tissues during surgical procedures, and the like. The cells included within the sample may be fresh, frozen, fixed in formalin or similar preservatives or may be embedded in paraffin or preserved by similar tissue archival procedures. With tissues fixed in formalin or similar preservatives, the samples may be washed with an appropriate solution, such as phosphate buffered saline, to remove residual preservative prior to DNA purification. In one method, formalin fixed samples are washed twice in 30 mL of phosphate buffered saline.

The DNA is preferably extracted from the sample and purified by any art conventional method. In one example, tissue is purified by means of a QIAamp® tissue kit for isolation and purification of nucleic acids or a Qiagen DNAeasy Kit (both supplied by Qiagen, Valencia, Calif.) using the manufacturer's suggested protocols. Alternatively, either frozen, finely powdered tissue or suspensions of washed cells can be mixed with a lysis agent, such as 5 volumes of lysis buffer (0.1M EDTA, 0.5% Sarkosyl, pH 8.0) and 20 μg/mL boiled RNAase at 55° C. in a shaking water bath for 30 minutes. DNA can then be extracted, such as by addition of Proteinase K (United States Biochemical Corp., Cleveland, Ohio) to 200 μg/mL and after a suitable time, such as 4 hours, extracting the mixture, such as extraction twice with 2.5 volumes of a 1:1 mixture of phenol and chloroform, and twice with 2.5 volumes of chloroform alone. The solutions containing DNA can then by dialyzed and placed in a suitable buffer, such as dialyzed against TE buffer (1 mM EDTA, 10 mM Tris HCl, pH 7.8), precipitated with ethanol, resuspended in TE, and stored at 4° C. It is to be appreciated that the method employed to extract and purify DNA from the sample, such as a tissue, may be any method producing DNA of suitable purity which is compatible with the methods of analysis.

Following extraction and purification of DNA by any method, to the extent that such extraction and purification is required for the DNA quantification method to be employed, the total DNA in the sample may then be quantitated. In one method, DNA is quantitated utilizing the fluorescent dye, PicoGreen® (Molecular Probes, Eugene, Oreg.), using a human genomic DNA as a standardized control, such as commercially available purified DNA peripheral blood lymphocytes (Promega, Madison, Wis.). PicoGreen is added to both the standard and purified DNA, and excited at 480 nm, as described by the supplier. Fluorescence emission intensities are measured at 520 nm using a Luminescence Spectrometer LS50 (Perkin Elmer, Boston, Mass.). Concentrations of the DNA samples are calculated from the equation describing the best-fit line generated with the standardized control DNA.

The assay and method described above utilize PicoGreen detection using a luminescence spectrometer, and in one embodiment with quantification of telomeric DNA using a film development and subsequence digitization of images. However, any of a number of detection methods may be employed. These include, without limitation, use of film development or a charge-coupled device (CCD) or other direct detection device with PicoGreen detection, using of radioactive markers for total DNA with detection by means of radiodetection, and the like. While PicoGreen is one commercially available fluorescent dye that is employed for DNA quantitation, other dyes, including other fluorescent dyes, are known in the art that may be employed for DNA detection. Other methods known in the art may also be employed for determination of total DNA, including methods employing radioactive measures and the like. Thus methods such as UV spectroscopy, weighing the DNA sample, and the like may be employed. In addition, in the practice of certain embodiments of the invention herein described, other markers for or correlates of total genomic DNA may be employed. As disclosed in U.S. Pat. No. 5,871,926, it is possible to employ centromere-specific probes. Thus it is possible to use centromere markers or markers for other conserved sequence areas, such as micro-satellites or ribosomal RNA, as a proxy for total genomic DNA in a sample.

Quantitation of Telomere DNA. In one preferred embodiment, telomere DNA is determined by hybridization with a telomere-specific probe with detection and quantification by means of chemiluminescent detection. Slot-blots are prepared as generally described in U.S. Pat. No. 5,871,926 and Bryant, J., K. Hutchings, R. Moyzis and J. Griffith: Measurement of telomeric DNA content in human tissues. BioTechniques 23:476-484 (1997). In brief, DNA is applied to Tropilon-Plus™ membranes (Tropix, Bedford, Mass.), air-dried and cross-linked with 1,200 mJ (UVP, Upland, Calif.). Following cross-linking, each blot is submerged in 0.25 M sodium phosphate buffer, pH 7.2. Each blot is prehybrized in a 200 mL glass hybridization bottle (Belico Glass, Vineland, N.J.) for one hour at 60° C. in 50 mL of hybridization buffer (7% SDS, 0.25M sodium phosphate buffer pH 7.2, 0.001M EDTA pH 8.0). Following prehybridization, the buffer is replaced with 50 mL of fresh hybridization solution containing 300 pmoles of telomere-specific, fluorescein 3′ end-labeled probe [5′ TTAGGG 3′]₄ (IDT, Coralville, Iowa). Each blot is hybridized at 60° C. for a minimum of 12 hours and a maximum of 16 hours. Following hybridization, the blot is washed twice at room temperature for 5 minutes in 30 mL of 2×SSC (0.3M NaCl, 0.03M Na₃C₆H₅O₇—2H₂O), 1% SDS. The initial wash is followed by two high temperature washes at 60° .C for 15 minutes in 30 mL of preheated 1×SSC, 1% SDS. Finally, the blot is washed twice at room temperature for 5 minutes in 30 mL of 1×SSC. All washes are performed in glass hybridization bottles with constant agitation in a Bellco AutoBlot hybridization oven (Bellco Glass, Vineland, N.J.).

After hybridization, telomere DNA is quantitated using the Southern Star™ chemiluminescent kit as described by the supplier (Tropix, Bedford, Mass.). The blots are equilibrated for 5 minutes in two successive 20 mL washes of blocking buffer [PBS (0.058M Na₂HPO₄, 0.017M NaH₂PO₄—H₂O, 0.068M NaCl), 2% I-block reagent (Tropix, Bedford, Mass.), 0.1% Tween-20 (Sigma, St Louis, Mo.)]. Each blot is then incubated separately for 45 minutes in 30 mL of blocking buffer at room temperature with constant agitation. The blocking buffer is discarded and replaced with 50 mL of blocking buffer containing 2 μL of alkaline phosphatase-conjugated, anti-fluorescein antibody (Tropix, Bedford, Mass.). The blot is incubated for 30 minutes at room temperature with constant agitation, washed in 30 mL of fresh blocking buffer, and then washed three times for 5 minutes at room temperature in 30 mL of wash buffer (PBS, 0.1% Tween-20) with constant agitation.

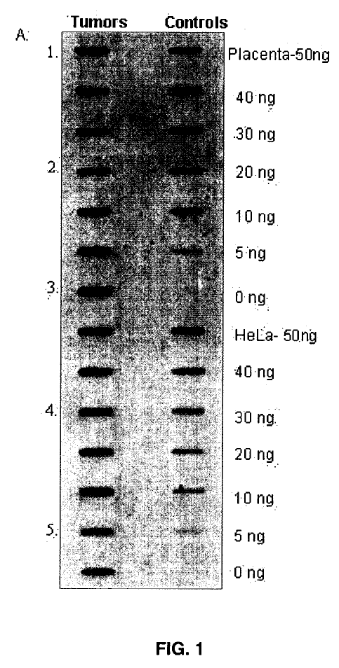

The pH of the blot is optimized for alkaline phosphatase activity by incubating the blot twice for two minutes in 20 mL of 1× assay buffer (Tropix, Bedford, Mass.). The surface of the blot is completely covered with approximately 4 mL of CDP-Star® chemiluminescent substrate (Tropix, Bedford, Mass.) and incubated for 5 minutes at room temperature. A typical slot blot showing placental, HeLa and five archival paraffin-embedded prostate tumor samples is shown in FIG. 1. Following incubation, CDP-Star® is wicked away, the blot sealed in a plastic bag and exposed to Hyperfilm (Amersham Pharmacia Biotech, Buckinghamshire England) for 2 or 5 minutes.

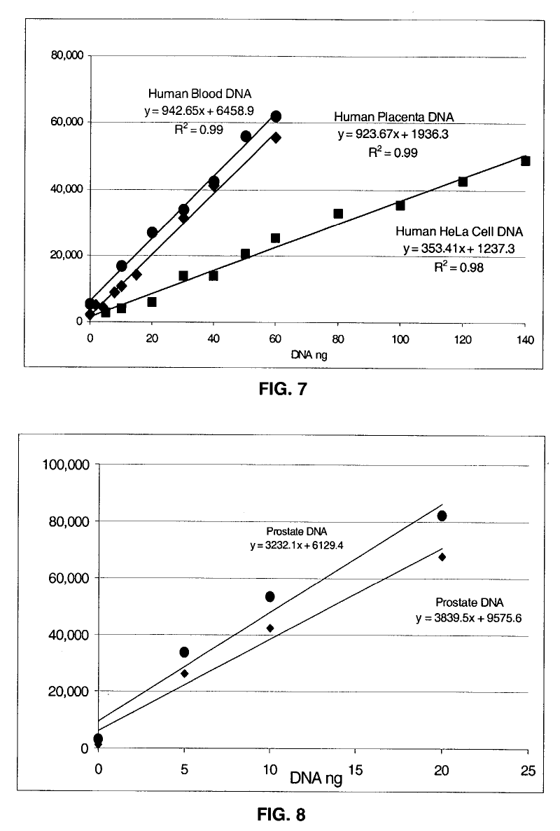

Films are developed (Konica Medical Film Processor-model QX-70) and optionally scanned (Hewlet-Packard ScanJet ADF). The intensity of the telomere hybridization signal can be determined from the digitized images, such as with Nucleotech Gel Expert Software 4.0 (Nucleotech, San Mateo, Calif.). Telomere content is determined by comparing the slope of the equation generated from DNA input versus relative signal from a standard to the samples at known concentration. Typical results are shown in FIG. 7, depicting representative data demonstrating differential telomere DNA contents in normal somatic (human blood lymphocytes and placenta) and tumor (HeLa) cells, and FIG. 8, depicting representative data demonstrating telomere DNA contents in sham needle core biopsies of prostate tumor DNA. Slopes, x-intercepts and correlation coefficients in FIGS. 7 and 8 were calculated by linear regression.

In general, any telomere-specific probe utilized with any detection system, including optionally an amplification and detection system, may be employed. Thus a CCD or other direct detection device may be employed for telomeric DNA detection, as well as methods incorporating UV detection, radionuclide detection, and the like. It is also possible and contemplated that other assay methods can be employed, including assay methods that yield results in substantially shorter time periods. Thus for example hybridization events may be detected by means of biosensing strategies that employ label-free detection of hybridization events. In one embodiment, an electrode-based detection method may be employed, including methods utilizing a target complementary DNA sequence in a polymeric film forming an electrode surface, with detection by electrochemical means. In other assay methods, a “molecular beacon” approach may be employed, including methods wherein a “quencher” is coupled to a fluorescent probe and complementary DNA sequence, such that hybridization may be detected.

In one embodiment, two different aliquots of purified and extracted DNA are employed, with one aliquot utilized for detection of total DNA, and another aliquot utilized for detection of telomeric DNA. However, it is also possible and contemplated that a single aliquot or quantity of DNA is employed for both detection of total DNA and telomeric DNA. In this embodiment it is preferable that detection methods be employed which are complementary, and that may be employed simultaneously or sequentially, on the same sample, with separate signals detectable for each of total DNA and telomeric DNA. For example, in one embodiment, two measures are made, one for telomeric segments and one for another segment that is conserved and may be correlated to total genomic DNA, such as centromeric segments. In one preferred embodiment, each target (i.e., a telomere and total DNA, a centromere or another marker) is associated with a fluorescent marker of different wavelength. The relative telomere content, as a function of total DNA, may thus be determined by the wavelength(s) emitted. Alternatively, one measure, such as of total DNA, may employ a fluorescent marker, while another measure, such as of telomeric DNA, may employ a different marker, such as a radioactive marker or a fluorescent marker of a different wavelength.

Determination of Telomeric DNA as a function of total DNA. Based on the measures of telomeric DNA and total DNA, it is possible to determine the telomeric DNA relative to total DNA by reference to a standard. Such comparison may be made to patient-specific normal telomere values, or to reference standards. Thus tissue may be obtained from a patient at a site distal to cancerous sites. In one embodiment, buccal brushing samples may be conveniently obtained, and teleomore values determined for the DNA content thereof. Comparisons may also be made to normalized or average patient populations, with adjustment for age, sex, race or other factors as appropriate.

In one embodiment, serial dilutions of purified DNA are employed for detection of both total DNA and telomeric DNA, and the slopes of each plot, together with slopes of such reference standards as is desired, are calculated. The resulting slopes thus provide a measure of each desired parameter. However, it is also possible to employ a form of single point analysis rather than utilizing multiple samples to generate a slope.

The experimental data demonstrates that there is a linear relationship (R²=0.98-99) between telomere DNA contents in samples of 5 to 120 ng of human genomic DNA purified from placenta, HeLa cells and peripheral blood lymphocytes using the methods of the invention. Telomere contents in the placenta and the peripheral blood lymphocyte DNA, representative of normal somatic cells, are nearly identical (slopes=923 vs 942 respectively). In contrast, the telomere content in HeLa DNA, representative of tumor cells, was significantly decreased (slope=353), as shown on FIG. 7. Telomere DNA contents in genomic DNA isolated from sham needle core biopsies ranged from 48-100% of the placental control, as shown on FIG. 8. There was no signal when equivalent amounts of a non-specific plasmid DNA (pGEM®-easy vector, Promega, Madison, Wis.) was substituted for genomic DNA in the assay.

Tumor and biopsy specimens typically contain mixtures of normal cells and tumor cells, whose telomeres differ in length. Thus, the observed telomere content of such a specimen reflects both the telomere contents and relative proportions of normal and tumor cells. The observed telomere content (TCobS) can be expressed quantitatively by the following equation (1):

TC_{obs}=(TC_{N})(% N)+(TC_{T})(% T) (1)

where TC_{N} and TC_{T} are the telomere contents in normal and tumor cells, respectively, and % N and % T are the percentages of normal and tumor cells in the specimen, respectively. Ideally, the telomere content of tumor cells in mixed samples is calculated by the following equation (2):

(TC_{T})=[TC_{obs}−(TC_{N})(% N)]/(% T) (2)

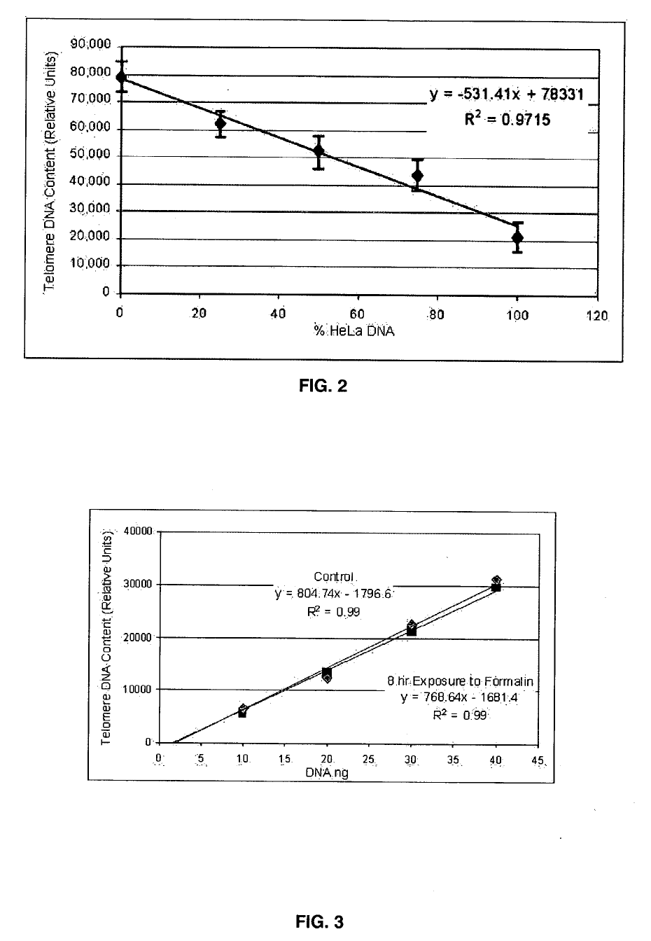

when TC_{obs}, TC_{N}, and % T (defined by histopathological examination) are known. To validate this approach, telomere contents were measured in mixtures containing defined proportions of HeLa cell DNA, mimicking tumor cells, and placental DNA, mimicking normal cells, as shown on FIG. 2. There was a linear and predictable relationship between the telomere contents and the relative proportions of HeLa and placenta DNA in each of the admixtures. Identical results were obtained when telomere contents were assayed in defined admixtures containing 30, 40, 50 or 60 ng of total genomic DNA. Thus the contributions of truncated cell telomeres in admixtures containing as much as 75% normal DNA can be defined.

Clinical Application of Telomere Determination. In analysis of tissues, it was discovered that decreased telomeric content is observed in the margins of tumors, including margins that by all other pathological evaluation techniques are normal tissue. Thus decreased telomeric content is an early marker for cells and cell populations that are pre-cancerous, or that may become cancerous cells identifiable by conventional pathological means. Thus by means of the assays and methods of this invention, or other methods to determine either telomere length or content, it is possible in surgical procedures and the like to delineate tissue that should be excised. The existence of otherwise normal tissue as determined by pathologic examination, but which tissue exhibits telomeric differences, including decreased telomeric content, is further a predictor of tumor metastases and tumor metastatic potential.

The methods and techniques described herein may be used with tumors other than prostate and breast cancers, and may in general be used with any cancer. Preliminary data demonstrates correlation to other tumors, thereby demonstrating that the methods and assays described herein have application for non-hormone dependent tumors.

The methods and techniques described herein may be employed for a number of purposes. In one embodiment, the assays described herein are performed on routine pap smears, as an additional predictor of cancer potential. Other sources of cells may be employed, such as for example cells obtained by nipple ravage. Traditional cell sources, such as biopsies, needle biopsies and the like, may similarly be employed.

The assays and methods described herein may be used for monitoring of progression of diseases other than cancer, and further for determination of the efficacy of therapy. For example, the assays and methods may be employed in HIV infection, to determine the telomere status of T-cells and T-cell subpopulations. By determining telomeric content and the rate of change of telomeric content over time the number and/or rate of cell division may be obtained. Such assays and methods may be used with any of a number of diseases that affect the rate of cell division of specific cell populations. Thus in addition to HIV disease, such assays and methods may be employed for other viral diseases, such as for example various forms of hepatitis.

A significant and substantial advantage of the present invention is that it may be employed with very small samples, as small as 2 to 5 ng of genomic DNA. This quantity of DNA is the approximate equivalent of less than about 700 cells, and thus is compatible with a needle biopsy or similar method of acquisition of patient tissue.

Currently available prognostic markers often fail to identify patients with lethal, metastatic tumors. Accordingly, cancer patients often receive empiric, aggressive therapies that, in fact, may not be necessary. Thus, there is a pressing need to identify more informative prognostic markers. As disclosed herein, it now appears that tumors with the shortest telomeres have the most aggressive phenotypes. It has further been discovered that telomere content may be evaluated as either “high” or “low” telomere content, such as by determining the median telomere content in a study population, and that such determination as high or low telomere content has significant prognostic value, and thus may be employed for determination of treatment plans, treatment options and the like. Thus in one study employing the detection methods described herein it was demonstrated that telomere content is associated with clinical outcome. One group was selected to contain women whose breast cancer tumor markers made it difficult to differentiate between patients with aggressive, lethal tumors and those with less aggressive disease, i.e. the women with the most problematic prognoses. In this group, telomere content predicted death from breast cancer or disease recurrence (p=0.01). To ensure that this result was not an artifact of the study population, the analysis was repeated on an overlapping, less homogeneous group. This group, nearly double the size of the initial population, was formed by the addition of randomly selected patients. In the expanded study group, telomere content again predicted death from breast cancer or disease recurrence (p=0.03).

In a second, independent study, the association of telomere content and outcome in prostate cancer was examined. Using a randomly selected prostate study population, it was demonstrated that telomere content predicted death from prostate cancer or disease recurrence (p=0.04). Telomere content was not associated with patients' ethnicity, age at diagnosis, tumor size or nodal involvement in either breast or prostate cancer, nor with estrogen- nor progesterone-receptor status in breast cancer, nor Gleason sum score nor seminal vesicle status in prostate cancer. Thus, telomere content is independent of these existing, prognostic tumor markers. Collectively, these data demonstrate that telomere content is an informative, independent and novel prognostic marker for breast and prostate cancer.

Several characteristics of the telomere content assay described herein make it well-suited for clinical use: it requires very small quantities of DNA that can be isolated from fresh, frozen or paraffin-embedded tissue; the results are not affected by DNA fragmentation; and, the data analysis is straight forward. Importantly, while breast and prostate tumors are typically heterogeneous, telomere content was independent of the fraction of normal cells in the tumor, precluding the requirement for microdissection of tissue.

The utility and application of the methods described herein may readily be applied to diseases such as cancer. For example, approximately 80% of premenopausal women diagnosed with breast cancer without nodal metastasis involvement will be free of disease for five years when treated with surgery and radiotherapy alone (Polychemotherapy for early breast cancer: an overview of the randomised trials. Early Breast Cancer Trialists' Collaborative Group. Lancet 352(9132):930-42 (1998)). Nonetheless, because currently available prognostic markers cannot differentiate between the 20% of tumors that will metastasize and the 80% that will not, the NIH/NCI and St. Gallen guidelines recommend adjuvant chemotherapy for 95% and 80% of these women, respectively. Goldhirsch, A., et al.: Meeting highlights: International Consensus Panel on the Treatment of Primary Breast Cancer. J Nat Cancer Inst, 90(21):1601-8 (1998); Glick, J. H., et al.: Meeting highlights: adjuvant therapy for primary breast cancer. J Natl Cancer Inst, 84(19):1479-85 (1992); Goldhirsch, A., et al.: Meeting highlights: International Consensus Panel on the Treatment of Primary Breast Cancer. Seventh International Conference on Adjuvant Therapy of Primary Breast Cancer. J Clin Oncol, 19(18):3817-27 (2001).

Women with lymph node-positive breast cancer typically receive adjuvant chemo- and radiation-therapies which extend disease free interval, but may not increase overall survival. Thus, there is a critical need to identify more informative prognostic markers. The data disclosed herein includes three independent analyses, comprising a total of 66 patients and 94 frozen and paraffin-embedded tissue specimens. The results demonstrate that reduced telomere content predicts breast cancer recurrence and disease-free survival in women with node positive breast cancer, independent of other common prognostic markers and adjuvant therapies. This data is consistent with, but not necessarily dependant upon, the hypothesis that dysfunctional telomeres, possibly by generating genetic instability, drive tumor progression to the phenotypic variability necessary for distant metastasis of breast cancers.

In addition to the presumably random genetic events caused by telomere loss, a recent study has demonstrated a second mechanism by which telomere shortening leads to phenotypic variability. Blackburn and colleagues have used microarray analysis to define a transcriptional profile in yeast named the telomere deletion response (TDR), that is expressed when yeast telomeres can no longer be maintained by telomerase (Nautiyal, S., J. L. DeRisi, and E. H. Blackburn: The genome-wide expression response to telomerase deletion in Saccharomyces cerevisiae. Proc Natl Acad Sci U S A, 99(14):9316-9321 (2002)). In addition to genes whose transcription is affected by DNA damage and environmental stress, including genes involved in glucose metabolism, the TDR includes a small set of genes, termed the telomerase deletion signature, whose expression is specific to telomere loss. The telomere deletion signature is characterized by an up-regulation of a variety of genes, including a cyclin dependent kinase activating kinase, a DNA binding protein and genes involved in ribosomal biogenesis. Although it has not yet been determined if a similar expression pattern exists in mammalian cells, it seems probable that telomere dysfunction induces specific changes in gene expression which might facilitate cell growth and survival in the metabolically demanding conditions of a growing tumor.

The invention disclosed herein also further demonstrates that telomere content in coexisting histologically normal breast tissues predict disease recurrence. While it is clear that tumor cells typically have reduced telomere content, most likely due to tumor cells' extensive proliferation, it is difficult to explain why telomeres would be reduced in coexisting histologically normal tissues. The lack of an association between telomere content and patients' age at diagnosis implies that reduced telomere content in coexisting histologically normal tissues is not the result of normal cellular aging. The increased cellular proliferation associated with desmoplasia also is unlikely to reduce telomeres in coexisting histologically normal tissues, since there is no apparent relationship between desmoplasia and telomere content in the specimens studied. Moreover, the coexisting histologically normal tissues were obtained from sites distal to the tumor margins (typically 1 to 5 cm) further precluding the contribution of desmoplasia to the reduced telomere content phenotype. Whatever the cause of the telomere loss observed in coexisting histologically normal tissues, the continued differentiation and proliferation of the breast in response to the onset of puberty, pregnancy or the menstrual cycle implies that genetic instability resulting from telomere dysfunction would be perpetuated and subject to clonal selection.

The novel discovery that reduced telomere content in coexisting histologically normal tissues is associated with disease recurrence and survival has several important implications. First, coexisting histologically normal cells typically contaminating tumor specimens will not diminish the prognostic value of the telomere content assay, thus precluding the necessity of microdissection. Second, the genetic events that influence a tumor's potential to produce aggressive disease may occur early in tumorigenesis, or independent of tumorigenesis, prior to phenotypic changes. It is possible that telomere content is reduced through the entire breast gland, or as recently suggested in a paper by Cawthen and coworkers, that there may be polymorphic differences in telomere length in different individuals (Cawthon, R. M., et al.: Association between telomere length in blood and mortality in people aged 60 years or older. Lancet 361(9355):393-5 (2003)).

Tumors developing within domains of dysfunctional telomeres, whatever the size of the domain, may have greater genetic instability and resulting phenotypic variability than tumors that arise within domains with more stable genomes. Thus it may be that that tumors developing in these domains will have a greater probability of containing cells capable of extravasation and metastasis: i.e., those that will result in poor patient outcome. Whatever the etiology or mechanism of action, it has now been demonstrated that a determination of telomere content in coexisting histologically normal tissues is of independent prognostic significance.

This invention thus discloses that telomere DNA content is a novel and independent prognostic marker in cancers, including breast, prostate and other cancers. The discovery that telomere content in coexisting histologically normal tissue also has prognostic value is novel and has important implications for tumor progression since it demonstrates that genetic changes in cell areas in which a tumor develops can be associated with tumor phenotype and patient outcome.

Industrial Applicability:

The invention is further illustrated by the following non-limiting examples.

### EXAMPLE 1

### DNA Isolation

DNA was purified from human placental tissue and cultured HeLa cells as generally described above. Waste frozen human prostate tissue was obtained from the University of New Mexico Solid Tumor Facility. Sham needle-core biopsies were produced from the frozen prostate tissue using a 2 cm long 18-gauge needle core. Pieces (0.2-0.4 g) of human placental tissue were submerged in formalin (Sigma, St Louis, Mo.) for 0, 15, 30, 60, 120, 240 and 480 minutes at room temperature. The tissue was washed twice in 30 mL of phosphate buffered saline to remove any residual formalin prior to DNA purification. DNA was purified from sham needle core biopsies and formalin-treated placenta using the Qiagen DNAeasy Kit and the manufacturer's protocol. The average yield of DNA from a 2 cm long, 18-gauge frozen needle core was 150 ng (n=9, SD 159 ng). DNA yield was not affected by formalin fixation. Archival paraffin-embedded prostatectomy tissue was obtained from the New Mexico Tumor Registry as approved by the Human Research Review Committee of the University of New Mexico. DNA was purified from archival paraffin embedded tissue utilizing the same general methods.

### EXAMPLE 2

### Quantification of DNA

Placenta, HeLa, sham needle core biopsy, and paraffin-embedded prostatectomy DNA was quantitated with the fluorescent dye, PicoGreen, using a commercially available human genomic DNA purified from peripheral blood lymphocytes (Promega, Madison, Wis.) as a standardized control. PicoGreen was added to both the standard and purified DNAs, and excited at 480 nm, as described by the supplier. Fluorescence emission intensities were measured at 520 nm using a Luminescence Spectrometer LS50 (Perkin Elmer, Boston,Mass.). Concentrations of the DNA samples were calculated from the equation describing the best-fit line generated with the standardized control DNA.

### Example 3

### Preparation and Hybridization of Slot Blots

Slot blots were prepared; briefly, DNA obtained by methods of Example 1 was applied to Tropilon-Plus™ membranes (Tropix, Bedford, Mass.), air-dried and cross-linked with 1,200 mJ (UVP, Upland, Calif.). Following cross-linking, each blot was submerged in 0.25M sodium phosphate buffer, pH 7.2. Each blot was prehybrized in a 200 ml glass hybridization bottle (Bellco Glass, Vineland, N.J.) for one hour at 60° C. in 50 mL of hybridization buffer (7% SDS, 0.25M sodium phosphate buffer pH 7.2, 0.001 M EDTA pH 8.0). Following prehybridization, the buffer was replaced with 50 mL of fresh hybridization solution containing 300 pmoles of telomere-specific, fluorescein 3′ end-labeled probe [5′ TTAGGG 3′]₄ (IDT, Coralville, Iowa). Each blot was hybridized at 60° C. for a minimum of 12 hours and a maximum of 16 hours. Following hybridization, the blot was washed twice at room temperature for 5 minutes in 30 mL of 2×SSC (0.3M NaCl, 0.03M Na₃C₆H₅O₇—2H₂O), 1% SDS. The initial wash was followed by two high temperature washes at 60° C. for 15-minute in 30 mL of preheated 1×SSC, 1% SDS. Finally, the blot was washed twice at room temperature for 5 minutes in 30 mL of 1×SSC. All washes were performed in glass hybridization bottles with constant agitation in a Bellco AutoBlot hybridization oven (Bellco Glass, Vineland, N.J.).

### Example 4

### Detection and Quantification of Telomere DNA

After hybridization as in Example 3, telomere DNA was quantitated using the Southern Star™ chemiluminescent kit as described by the supplier (Tropix, Bedford, Mass.). The blots were equilibrated for 5 minutes in two successive 20 mL washes of blocking buffer [PBS (0.058M Na₂HPO₄, 0.017M NaH₂PO₄—H₂O, 0.068M NaCl), 2% I-block reagent (Tropix, Bedford, Mass.), 0.1% Tween-20 (Sigma, St. Louis, Mo.)]. Each blot was then incubated separately for 45 minutes in 30 mL of blocking buffer at room temperature with constant agitation. The blocking buffer was discarded and replaced with 50 mL of blocking buffer containing 2 μL of alkaline phosphatase-conjugated, anti-fluorescein antibody (Tropix, Bedford, Mass.). The blot was incubated for 30 minutes at room temperature with constant agitation, washed in 30 mL of fresh blocking buffer, and then washed three times for 5 minutes at room temperature in 30 mL of wash buffer (PBS, 0.1% Tween-20) with constant agitation.

The pH of the blot was optimized for alkaline phosphatase activity by incubating the blot twice for two minutes in 20 mL of 1× assay buffer (Tropix, Bedford, Mass.). The surface of the blot was completely covered with approximately 4 mL of CDP-Star® chemiluminescent substrate (Tropix, Bedford, Mass.) and incubated for 5 minutes at room temperature, resulting in a blot as depicted in FIG. 1. Following incubation, CDP-Star® was wicked away, the blot was sealed in a plastic bag and exposed to Hyperfilm (Amersham Pharmacia Biotech, Buckinghamshire England) for 2 or 5 minutes.

Films were developed (Konica Medical Film Processor-model QX-70) and scanned (Hewlet-Packard ScanJet ADF). The intensity of the telomere hybridization signal was determined from the digitized images with Nucleotech Gel Expert Software 4.0 (Nucleotech, San Mateo, Calif.). Telomere content was determined by comparing the slope of the equation generated from DNA input versus relative signal from the placental standard to the samples at known concentration, as shown in FIG. 7.

### Example 5

### Telomere DNA Content In Formalin Fixed Tissue

It is not uncommon for surgical and biopsy specimens to be stored in formalin for as long as 8 hours prior to embedding in paraffin. To determine whether formalin fixation affected the reliability of the telomere DNA content assay, human placenta was submerged in formalin for up to 8 hours at room temperature prior to DNA purification by the method of Example 1, with results as shown in FIG. 3. Telomere DNA contents measured in genomic DNA isolated from tissue treated with formalin were not different from the untreated control (slopes=768, 804 respectively). Telomere DNA content could also be assayed with DNA purified from paraffin embedded archival prostatectomy specimens, as shown in FIG. 8.

### Example 6

### Study Populations for Breast Cancer Analysis

Specimens and associated patient data were identified by the New Mexico Tumor Registry (NMTR). The NMTR database links anonymous patient histories to archival cancer specimens through pathology reference numbers and dates of procedures in accord with all federal regulations, as approved by the University of New Mexico Health Science Center Human Research Review Committee. Three groups of tissues, including tumor and coexisting histologically normal tissues, from two independent patient cohorts, were used for this investigation. The first cohort was comprised of 38 randomly selected frozen specimens of invasive breast carcinoma (Table 1). In most instances, patients' records included age at diagnosis, tumors' sizes, grades, axial lymph node involvement, the fraction of cells in S-phase and at least 48 months of follow up (median 96 months). The second case-control cohort involved 28 women who had large, node positive cancers and received mastectomies prior to 1994. Evaluable data typically included patients' ethnicities, ages at diagnosis, tumor's size and grade and axial lymph node involvement. Estrogen and progesterone receptor status was also available for approximately half of the tumors. Patients in the second group had been followed for a minimum of 84 months or until documented disease recurrence (usually death from metastatic breast cancer). Thirteen women received adjuvant hormone therapy, 15 received adjuvant chemotherapies and 10 women received both. Formalin-fixed, paraffin-embedded archival tumor and coexisting histologically normal tissues were utilized in the second study.

Paraffin-embedded and frozen tissue sections were stained with hematoxylin and eosin and were examined microscopically. Tumor tissues typically contained from 75-100% tumor cells. Nearly all of the paraffin-embedded tissues were obtained from women who were treated with partial or full mastectomy. It was not possible to determine the distance between the sites from which the tumor and coexisting histologically normal tissues were derived. However, since coexisting histologically normal tissues were always cut from different paraffin embedded blocks, usually from the nipple, it may be inferred that the coexisting histologically normal tissue was at least 1 to 5 cm from the tumor margin.

### Example 7

### DNA Isolation In Breast Cancer Studies

Frozen tissues were cut into small pieces using a sterile scalpel. DNA was then extracted using the Qiagen DNAeasy Kit following the manufacture's protocol. For paraffin-embedded tissues, DNA was extracted from twelve, serial 25 μm-thick sections. Tissue sections,were deparaffinized in octane and washed in ethanol. DNA was then purified using the Qiamp Tissue Kit (Qiagen, Valencia Calif.) as in Example 1. DNAs were quantitated with the fluorescent dye, PicoGreen, following the manufacturer's protocol as in Example 2. Placentaland HeLa DNA controls, with telomere restriction fragments lengths of 10.1 and 5.4 KB, respectively, were purified as and used as controls in the TC assay.

### Example 8

### Telomere Content Assay for Breast Cancer Analysis

Slot blots were prepared as described above. Briefly, DNA was denatured, neutralized, loaded and fixed to Tropilon-Plus™ membranes. A telomere specific oligonucleotide end-labeled with fluorescein, (5′ TTAGGG 3′)₄-FAM, was hybridized to genomic DNA. Following hybridization, the blots were washed to remove non-hybridizing oligonucleotides. Telomere-specific oligonucleotides were detected by an alkaline phosphatase-conjugated anti-fluorescein antibody that produces light when incubated with the CDP-Star substrate. Blots were exposed to Hyperfilm for 2-5 minutes and digitized. The intensity of the telomere hybridization signal was determined from the digitized images with Nucleotech Gel Expert Software 4.0. Telomere content was expressed as a percentage of the average chemiluminescent signal of three replicate tumor DNAs compared to the value of the placental standard at the same genomic DNA concentration. Each tumor or coexisting, histologically-normal DNA was typically analyzed independently three times. In addition to placental DNA, DNA purified from HeLa cells was also included on each blot. The reproducibility of each experiment was verified by comparing telomere content of the HeLa DNA to telomere content of the placental DNA.

### Example 9

### Statistical Analysis for Breast Cancer Study

Associations between variables and TC groups (high and low) were assessed by chi-square analysis at the 95% confidence level. Similarly, odds ratios were calculated using a 95% confidence interval. The Log Rank Test with Kaplan-Meier graph was used to assess the relationship between telomere content groups (low and high) and the duration of disease-free survival. Wilcoxon Rank Sums Test was used to assess the relationship between patient outcome group (recurrence or disease-free) and telomere content as a continuous variable. Multivariate survival analysis was performed by the Cox Proportional Hazards Regression method.

### Example 10

### Relationship of TC to Disease Recurrence in Randomly Selected Breast Tumors

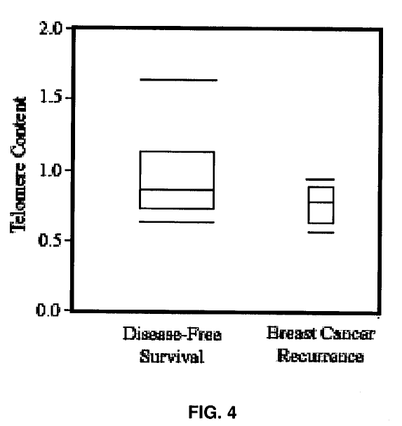

Telomere content was measured in 38 randomly selected, frozen breast tumors, including 8 tumors from women who subsequently developed recurrent disease. As shown in FIG. 4, the median and mean telomere contents of the tumors from patients that developed recurrent disease (75% and 75%, respectively) were less than the median and mean telomere contents of all tumors (84% and 95%, respectively) and the subset of tumors that did not recur (94% and 100%, respectively).

To evaluate possible relationships between telomere content and several common clinical parameters, the 38 tumors were divided into two groups: one with telomere content below the mean and the other with telomere content above the mean of the 38 tumors (“low” and “high” telomere content, respectively). As shown in Table 1, telomere content was not associated with patients' ages at diagnosis, tumors' sizes or grades, metastasis to axial lymph nodes, estrogen- or progesterone-receptor status, ploidy, or the fraction of S-phase cells. In contrast, the relationship between breast-cancer recurrence and telomere content group was significant at the 95% confidence level (p=0.03).

For comparison, the association between recurrence and other common prognostic factors was also investigated. When the tumors were divided into two groups based on recurrence (Table 1) there was no significant relationship with patients' ages at diagnosis, tumors' sizes or grades, presence of estrogen- or progesterone-receptors, ploidy, or fraction of S-phase cells. However, the relationship between recurrence and axial lymph node metastasis approached statistical significance (p=0.09), consistent with the widely accepted usage of axial node metastasis as a predictor of disease recurrence. Table 1 presents a telomere content analysis of frozen breast tumors.

TABLE 1

| | | Age@ | | | | | | | | | | | | | | | % S- | | | |
|---|---|---|---|---|---|---|---|---|---|---|---|---|---|---|---|---|---|---|---|---|
| | | DX | | Size¹ | | Grade² | | | Node³ | | ER⁴ | | PR⁵ | | Ploidy⁶ | | phase⁷ | | Recurrence⁸ | |
| | N | ≦50 | >50 | S | L | 1 | 2 | 3 | N | Y | N | Y | N | Y | D | A | L | H | N | Y |
| Low TC | 23 | 8 | 14 | 5 | 15 | 1 | 6 | 6 | 10 | 10 | 8 | 13 | 8 | 12 | 4 | 19 | 6 | 12 | 13 | 8 |
| High TC | 15 | 8 | 4 | 1 | 10 | 1 | 5 | 8 | 3 | 10 | 3 | 7 | 5 | 5 | 5 | 10 | 3 | 6 | 13 | 0 |

| p-value | | 0.18 | | 0.55 | | 0.68 | | | 0.24 | | 0.97 | | 0.90 | | 0.46 | | 1.00 | | 0.03 |
|---|---|---|---|---|---|---|---|---|---|---|---|---|---|---|---|---|---|---|---|
| Recurred N | 26 | 11 | 13 | 4 | 19 | 1 | 6 | 17 | 12 | 13 | 8 | 13 | 9 | 11 | 5 | 21 | 6 | 14 | |
| Recurred Y | 8 | 4 | 4 | 2 | 5 | 1 | 2 | 5 | 0 | 6 | 3 | 5 | 3 | 5 | 2 | 6 | 2 | 3 | |

| | | | | | | | | |
|---|---|---|---|---|---|---|---|---|
| p-value | 1.00 | 0.92 | 1.00 | 0.09 | 1.00 | 1.00 | 1.00 | 1.00 |

In Table 1, the following notes apply: 1. Tumor size: Small (S) <2 cm, Large (L): >2 cm in largest dimension; 2. Histopathological grade; 3. Metastasis to axial lymph nodes: No (N), Yes (Y); 4. Presence of estrogen receptor: No (N), Yes (Y); 5. Presence of progesterone receptor: No (N), Yes (Y); 6. Ploidy: Diploid (D), Aneuploid (A); 7. Percentage of S-phase cells: Low (L) <6.7%, High (H) >6.7%; and, 8. Disease recurrence: No (N); Yes (Y). The statistical significance (p) was calculated by chi-square at the 95% confidence level. Abbreviations used are: N, number; DX, diagnosis. Complete clinical information was not available for all patients.

### Example 11

### Telomere Content in Breast Tumors Predicts Disease-free Survival

In order to better define the relationship between telomere content and clinical outcome in breast cancer, a second independent study was designed to test the hypothesis that telomere content has prognostic value that is independent of large tumor size and metastasis to axial lymph nodes. Because the telomere content assay is not affected by DNA breakage and can be performed with as little as 5-10 ng of genomic DNA, it is well-suited for investigations with paraffin-embedded archival tissues. The retrospective study group was comprised of paraffin-embedded tumor and coexisting histologically normal tissues from 28 women with invasive breast tumors who received partial or radical mastectomies between 1982 and 1993, and who had been followed until documented disease recurrence (usually death from metastatic breast cancer) or for at least 84 months, with results as shown in FIG. 4. The characteristics of the study group are summarized in Table 2. In contrast to the first study population, in which roughly one third of the women had tumors which did not metastasize to the lymph nodes, approximately 90% of the tumors for which complete information was available had metastasized to the axial lymph nodes by the time of diagnosis (22/23), were large (15/17), or both (15/17). However, the study population also was selected to contain approximately equal numbers of tumors from women who remained disease free for at least 84 months after surgery (control group) and women who developed distant metastases within 84 months of their surgeries (case group). Thus, the study design permitted a critical assessment of the relationship between telomere content and disease recurrence, independent of the contributions of large tumor size and lymph node metastasis.

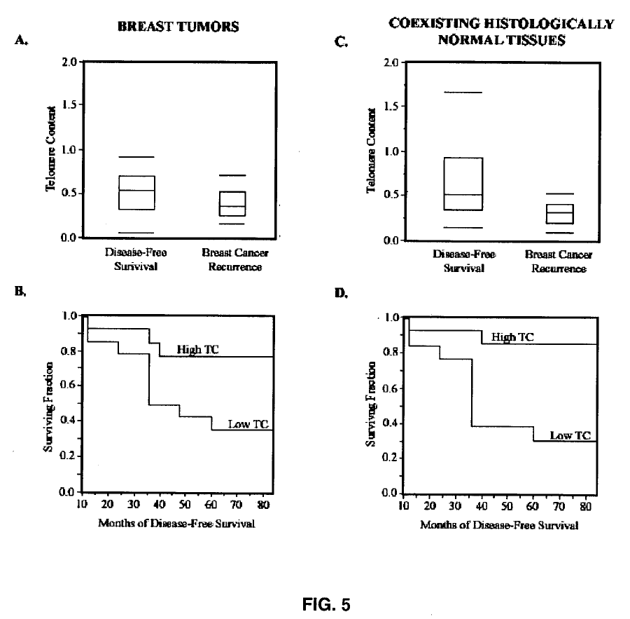

Telomere content was measured successfully in 25/28 of the tumor specimens. Median telomere content was used to divide the study population into two equal size groups (“high” and “low” telomere content). The relationships between telomere content groups and the several available clinical parameters were then evaluated. Consistent with the results obtained in the study of Example 10, there was no significant association between telomere content and patients' ethnicity, age at diagnosis, tumors' grades, or estrogen- or progesterone-receptor status. Nine of the 12 women in the low telomere content group had developed recurrent disease within 84 months of surgery. In contrast, 10 of 13 women in the high telomere content group remained disease-free for at least 84 months (p=0.03). Indeed, low telomere content was associated with a 10.0 (95% Cl: 1.2-105.6) odds ratio for disease recurrence. Moreover, Log Rank analysis (FIG. 5-B) indicated a highly significant association between the telomere content group and the length of disease-free survival (p=0.012). To further investigate the relationship between telomere content and disease-free survival, Wilcoxon rank sums analysis, in which telomere content is a continuous variable, was also performed. When tumors were grouped by clinical outcome, i.e. patients whose disease recurred within 84 months and those that remained disease-free (FIG. 5-A), Wilcoxon Rank Sums Test indicated a highly significant association between clinical outcome group and telomere content (p=0.013).

In order to determine if there was a relationship between breast cancer recurrence or overall survival and other markers, patients were divided into two groups based on clinical outcome (84 month disease-free survival vs. recurrent breast cancer). In this analysis there was no association between patients' clinical outcomes and ethnicities, ages at diagnosis, tumors' grade, or estrogen- or progesterone-receptor status (Table 2). Collectively, these data suggest that telomere content in breast tumors is an independent prognostic tumor marker. Table 2 thus presents a telomere content analysis of paraffin-embedded breast tumors.

TABLE 2

| | | Ethnicity¹ | | | Age@ DX | | Size² | | Grade³ | | | Node⁴ | | ER⁵ | | PR⁶ | | Recurrence⁷ | |
|---|---|---|---|---|---|---|---|---|---|---|---|---|---|---|---|---|---|---|---|
| | N | A | H | O | ≦50 | >50 | S | L | 1 | 2 | 3 | N | P | N | P | N | P | N | Y |
| Low TC | 12 | 7 | 4 | 0 | 9 | 3 | 1 | 9 | 2 | 5 | 5 | 0 | 11 | 3 | 5 | 2 | 6 | 3 | 9 |
| High TC | 13 | 7 | 4 | 1 | 10 | 3 | 1 | 6 | 2 | 6 | 4 | 1 | 11 | 1 | 5 | 1 | 5 | 10 | 3 |

| p-value | | 1.00 | | | 1.00 | | 1.00 | | 1.00 | | | 1.00 | | 0.80 | | 1.00 | | 0.03 |
|---|---|---|---|---|---|---|---|---|---|---|---|---|---|---|---|---|---|---|
| Recurred N | 13 | 7 | 5 | 0 | 11 | 2 | 2 | 6 | 3 | 4 | 5 | 1 | 11 | 1 | 5 | 1 | 5 | |
| Recurred Y | 12 | 7 | 3 | 1 | 8 | 4 | 0 | 9 | 1 | 7 | 4 | 0 | 11 | 3 | 5 | 2 | 6 | |

| | | | | | | | |
|---|---|---|---|---|---|---|---|
| p-value | 0.90 | 0.56 | 0.40 | 1.0 | 1.0 | 0.8 | 1.0 |

In Table 2, the following notes apply: 1. Ethnicity: Non-Hispanic White (A), Hispanic (H), Other (0); 2. Tumor size: Small (S) <2 cm, Large (L) >2 cm in largest dimension; 3. Histopathological grade; 4. Metastasis to axial lymph nodes: No (N), Yes (Y); 5. Presence of estrogen receptor: No (N), Yes (Y); 6. Presence of progesterone receptor: No (N), Yes (Y); and, 7. 84-month disease-free survival. The statistical significance (p) was calculated by chi-square at the 95% confidence level. Abbreviations are as in Table 1. Complete clinical information was not available for all patients.

### Example 12

### Telomere Content in Histologically-Normal Tissue Coexisting with Breast Tumors Predicts Clinical Outcome

Preliminary investigations suggested that it was not necessary to microdissect tumors to remove histologically normal cells when analyzing telomere content. In order to more fully investigate the relationship between telomere content in breast tumors and in coexisting histologically normal tissue, telomere content was measured in coexisting histologically normal tissues derived from the case-control study group. Telomere content was measured successfully in 27/28 of the samples. There was not a direct relationship between telomere content in tumor tissue and coexisting histologically normal tissue. However, when the median telomere content was used to divide the tumor and coexisting histologically normal issues into high and low telomere content groups, there was a statistically significant relationship between telomere content in tumor and coexisting histologically normal tissues (p=0.03). Nine of 13 women in the low telomere content group had recurrent disease within 84 months of surgery. In contrast, twelve of the fourteen women in the high telomere content group remained disease free for at least 84 months following surgery (p=0.01). Low telomere content was associated with a 13.5 (95% Cl: 1.6-153.6) odds ratio for disease recurrence. Log Rank analysis, as shown in FIG. 5-D, indicated a highly significant association between telomere content group and the length of disease-free survival (p=0.005). As described previously for the tumor tissues in Example 11, telomere content of the coexisting histologically normal tissues was also analyzed as a continuous variable. Again, telomere content in coexisting histologically normal was associated with patient outcome (p=0.03). Collectively, these data demonstrate that telomere content in breast coexisting histologically normal tissue is a novel prognostic tumor marker and indicate that microdissection of a breast tumor is not necessary to obtain meaningful prognostic data with the slot blot assay.

### Example 13

### Telomere DNA Content and Prostate Cancer

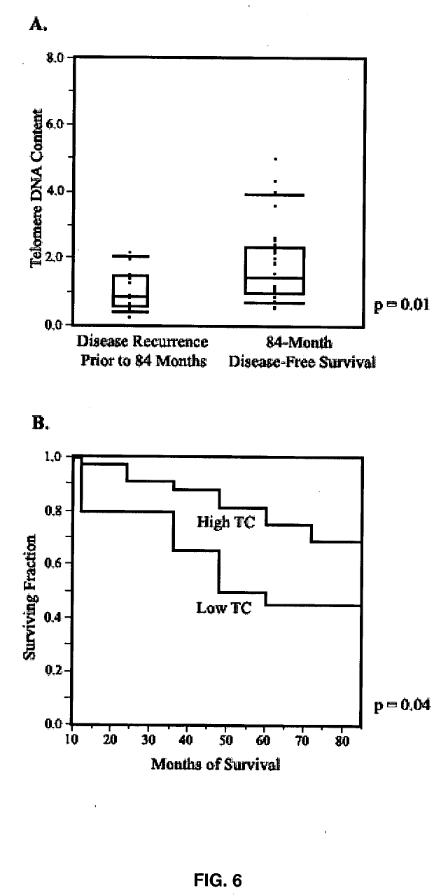

Using a carefully matched, limited prostate cancer study group (analogous to the initial breast cancer study group of Example 11) it was demonstrated that reduced telomere content was also associated with disease recurrence and death. This result, in combination with results for breast cancer described above, demonstrates that telomere content has prognostic significance in cancers with both a variety of markers and different tissues of origin. Telomere content was analyzed in paraffin-embedded prostate tumors from 51 randomly selected men. Twenty-one of these men developed recurrent prostate cancer (including 13 who died from prostate~cancer) within 84 months of surgery. The remaining 30 men remained disease-free for at least 84 months. The characteristics of the prostate study group are summarized in Table 3. As with breast tumors, prostate tumors were stratified by the median telomere content. Similarly, there was no association between telomere content and ethnicity, age at diagnosis, Gleason sum score or lymph node or seminal vesicle status. Reduced telomere content was again associated with patients who either died from, or developed, recurrent cancer within 84 months of surgery. FIG. 6-A, p=0.01. Moreover, Kaplan-Meier analysis demonstrated yet again that telomere DNA content was associated with clinical outcome. FIG. 6-B, p=0.04.

TABLE 3

| | | | | Age at | | Lymph | | Seminal | | Gleason | |
|---|---|---|---|---|---|---|---|---|---|---|---|
| | Ethnicity¹ | | | Diagnosis | | Nodes⁴ | | Vesicles⁴ | | Sum Score | |
| N | A | H | O | M² | R³ | Pos | Neg | Pos | Neg | M⁵ | R⁶ |
| 51 | 28 | 12 | 2 | 67 | 53-76 | 6 | 39 | 28 | 8 | 7 | 2-9 |

Characteristics of the prostate study group are shown. In Table 3, the following notes apply: 1. Patients' ethnicities were self-reported and classified as Anglo (A), Hispanic (H), or Other (0); 2. Median age; 3. Range of age of diagnoses; 4. Tumors' lymph node, seminal vesicle status; 5. Median Gleason sum score; and, 6. Range Gleason sum score.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. An assay for measuring telomere content as a function of total DNA in a sample of DNA, the assay comprising quantitating telomere content and total DNA content of the sample, and calculating the amount of telomere present relative to total DNA.

2. The assay of claim 1, wherein quantitating the total DNA content of the sample comprises adding a fluorescent dye capable of forming a fluorescent dye-DNA complex to the sample, measuring the amount of bound fluorescent dye, and correlating the amount of measured bound fluorescent dye relative to a control of known total DNA to determine total DNA.

3. The assay of claim 1, wherein quantitating telomere content comprises adding a labeled probe having a sequence complementary to a telomere repeat sequence to the sample, and measuring the amount of labeled probe.

4. The assay of claim 1, wherein the assay comprises a slot-blot assay.

5. The assay of claim 4, wherein slot-blotted DNA is hybridized with a telomere-specific oligonucleotide probe.

6. The assay of claim 1, wherein the DNA is obtained from paraffin-fixed tissue.

7. The assay of claim 1, wherein the DNA is obtained from formalin-fixed tissue.

8. The assay of claim 1, wherein the sample of DNA comprises less than about 50 ng of DNA.

9. The assay of claim 8, wherein the sample of DNA comprises less than about 10 ng of DNA.

10. The assay of claim 9, wherein the sample of DNA comprises less than about 5 ng of DNA.

11. A method for measuring telomere content as a function of total DNA in a sample including DNA, the method comprising the steps of:
adding a fluorescent dye capable of forming a fluorescent dye-DNA complex to the sample;
measuring the amount of bound fluorescent dye;
correlating the amount of measured bound fluorescent dye relative to a control of known total DNA to determine total DNA in the sample;
adding a labeled probe having a sequence complementary to a telomere repeat sequence to the sample;
measuring the amount of labeled probe; and
correlating the amount of bound probe measured relative to total DNA.

12. The method of claim 11, wherein the fluorescent dye is added to a first quantity of the sample and the labeled probe is added to a second quantity of the sample.

13. A method for measuring telomere content as a function of total DNA in a sample including DNA, the method comprising the steps of:
adding a fluorescent dye forming a fluorescent dye-DNA complex to two or more diluted quantities of the sample and separately to at least one first quantity of a standard including a known amount of DNA;
measuring the amount of bound fluorescent dye in the two or more diluted quantities of the sample and the at least one first quantity of standard;
determining the total DNA in the sample by comparing the fluorescent intensity of the diluted quantities of the sample to the at least one quantity of the standard including a known amount of DNA;
adding a labeled probe having a sequence complementary to a telomere repeat sequence to two or more quantities of the sample and separately to at least one second quantity of a standard including a known telomere content as a function of total DNA;
measuring the amount of labeled probe in the two or more diluted quantities of the sample and the at least one second quantity of standard; and
determining telomere content in the sample as a function of total DNA by comparing the fluorescent intensity of two or more quantities of the sample as a function of total DNA to the fluorescent intensity of the at least one second quantity of standard.

14. A method for measuring telomere content of tumor cells in a first sample including DNA from a mixed population of cells including tumor cells and histologically normal cells, comprising the steps of:
determining the telomere content of the first sample as a function of total DNA;
obtaining a second sample of histologically normal cells and determining the total telomere content of the second sample as a function of total DNA;
determining the percentage of the total cells of the first sample that are histologically normal;
calculating the telomere content of tumor cells in the first sample by means of the formula:
(TC_{T})=[TC_{obs}−(TC_{N})(% N)]/(% T)
wherein TC_{T} is the telomere content of the tumor cells of the first sample as a function of total DNA, TC_{obs} is the determined total telomere content of the first sample as a function of total DNA, TC_{N} is the determined telomere content in the second sample of histologically normal cells, % N is the percentage of histologically normal cells in the first sample and % T is the percentage tumor cells in the first sample.

15. The method of claim 14, wherein the total telomere content of the first sample as a function of total DNA is determined by quantitating telomere content and total DNA content of the first sample, and calculating the amount of telomere present relative to total DNA.

16. The method of claim 14, wherein the first sample and the second sample are obtained from the same patient.

17. A method for determining the prognosis of a patient with cancer, the method comprising obtaining a tumor specimen from the patient, determining the telomere content of the tumor specimen as a function of total DNA, and categorizing the determined telomere content with respect to a mean telomeric content.

18. The method of claim 17, wherein determining the telomere content of the tumor specimen as a function of total DNA further comprises purifying the sample to isolate DNA, quantitating telomere content and total DNA content of the sample, and calculating the amount of telomere present relative to total DNA.

19. The method of claim 18, wherein quantitating the total DNA content of the sample comprises adding a fluorescent dye capable of forming a fluorescent dye-DNA complex to the isolated DNA, measuring the amount of bound fluorescent dye, and correlating the amount of measured bound fluorescent dye relative to a control of known total DNA to determine total DNA.

20. The method of claim 18, wherein quantitating telomere content comprises adding a labeled probe having a sequence complementary to a telomere repeat sequence to the isolated DNA, and measuring the amount of labeled probe.

21. The method of claim 17, wherein the isolated DNA comprises less than about 50 ng of DNA.

22. The method of claim 21, wherein the isolated DNA comprises less than about 10 ng of DNA.

23. The method of claim 22, wherein the isolated DNA comprises less than about 5 ng of DNA.

24. The method of claim 17, wherein the cancer is a member selected from the group consisting of breast cancer and prostate cancer.

25. A method for determining the prognosis of a patient with cancer, the method comprising obtaining a coexisting histologically normal tissue sample from the patient and determining the telomere content of the coexisting histologically normal sample as a function of total DNA.

26. The method of claim 25, further comprising categorizing the determined telomere content of the coexisting histologically normal tissue with respect to a mean telomeric content.

27. The method of claim 25, wherein determining the telomere content of the coexisting histologically normal tissue sample as a function of total DNA further comprises purifying the sample to isolate DNA, quantitating telomere content and total DNA content of the sample, and calculating the amount of telomere present relative to total DNA.

28. The method of claim 25, wherein the coexisting histologically normal tissue sample is obtained from a site at least about 1 cm distal from any histologically abnormal tissue.

29. The method of claim 25, wherein the coexisting histologically normal tissue sample is obtained from a site within about 5 cm of histologically abnormal tissue.

30. The method of claim 27, wherein quantitating the total DNA content of the sample comprises adding a fluorescent dye capable of forming a fluorescent dye-DNA complex to the isolated DNA, measuring the amount of bound fluorescent dye, and correlating the amount of measured bound fluorescent dye relative to a control of known total DNA to determine total DNA.

31. The method of claim 27, wherein quantitating telomere content comprises adding a labeled probe having a sequence complementary to a telomere repeat sequence to the isolated DNA, and measuring the amount of labeled probe.

32. The method of claim 25, wherein the coexisting histologically normal tissue sample comprises less than about 50 ng of DNA.

33. The method of claim 32, wherein the coexisting histologically normal tissue sample comprises less than about 10 ng of DNA.

34. The method of claim 33, wherein the coexisting histologically normal tissue sample comprises less than about 5 ng of DNA.

35. The method of claim 25, wherein the cancer is a member selected from the group consisting of breast cancer and prostate cancer.

36. A method of determining tissue boundaries in a surgical procedure to resect cancerous or pre-cancerous tissues in a patient, comprising determining the telomere content of tissue as a function of total DNA.

37. The method of claim 36, wherein determining the telomere content of tissue as a function of total DNA further comprises purifying the tissue to isolate DNA, quantitating telomere content and total DNA content of the tissue, and calculating the amount of telomere present relative to total DNA.

38. The method of claim 37, wherein quantitating the total DNA content of the tissue comprises adding a fluorescent dye capable of forming a fluorescent dye-DNA complex to the isolated DNA, measuring the amount of bound fluorescent dye, and correlating the amount of measured bound fluorescent dye relative to a control of known total DNA to determine total DNA.

39. The method of claim 37, wherein quantitating telomere content comprises adding a labeled probe having a sequence complementary to a telomere repeat sequence to the isolated DNA, and measuring the amount of labeled probe.

40. The method of claim 37, wherein the cancer is a member selected from the group consisting of breast cancer and prostate cancer.
